(19) United States
(12) Patent Application Publication
(10) Pub. No.: US 2023/0225988 A1
(43) Pub. Date: Jul. 20, 2023

(54) ANTIVIRAL USE OF CALIXARENES

(71) Applicant: CEBINA GmbH, Vienna (AT)

(72) Inventors: Eszter NAGY, Vienna (AT); Gabor NAGY, Sopron (HU); Valeria SZIJARTO, Wien (AT); Robert KONRAT, Vienna (AT)

(21) Appl. No.: 18/011,154

(22) PCT Filed: Jun. 18, 2021
(86) PCT No.: PCT/EP2021/066558

§ 371 Date: Dec. 16, 2022

(30) Foreign Application Priority Data
Jun. 18, 2020 (EP) 20180838.3

(51) Int. Cl. A61K 31/05; A61P 31/14

(52) U.S. Cl. CPC A61K 31/05; A61P 31/14

(57) ABSTRACT

A calixarene compound in an antiviral effective amount for use as an antiviral substance in a pharmaceutical preparation for use in prophylactic or therapeutic treatment of a disease condition which is caused by or associated with an infection with a virus.

### FIELD OF THE INVENTION

The present invention relates to novel uses of a calixarene compound for treating viral infections, particularly methods and compounds for treating coronaviridae infections (e.g., SARS virus or MERS virus), influenza infections, human Respiratory Syncytial Viral infections and adenovirus infections.

### BACKGROUND OF THE INVENTION

Respiratory viruses are the frequent causative agents of disease in humans, with significant impact on morbidity and mortality worldwide. The respiratory viruses that most commonly circulate in all continents as endemic or epidemic agents are coronavirus, influenza virus, Respiratory Syncytial Virus (RSV), parainfluenza viruses, metapneumovirus, rhinovirus, adenoviruses, and bocaviruses. In addition to infection by single virus species, co-infection of different respiratory viruses is often seen. Coronaviruses are single-stranded RNA viruses, about 120 nanometers in diameter. They are prone to mutation and recombination and are therefore highly diverse. There are about 40 different varieties and they mainly infect human and non-human mammals and birds. They reside in bats and wild birds, and can spread to other animals and hence to humans.

There are four main genera (alpha, beta, gamma, and delta-coronavirus) based on their genomic structure. Alpha- and beta-coronaviruses infect only mammals, usually causing respiratory symptoms in humans and gastroenteritis in other animals. Until December of 2019, only six different coronaviruses were known to infect humans. Four of these (HCoV-NL63, HCoV-229E, HCoV-OC43 and HKU1) usually caused mild common cold-type symptoms in immunocompetent people and the other two have caused pandemics in the past two decades. In 2002-2003, the severe acute respiratory syndrome coronavirus (SARS-CoV) caused a SARS epidemic that resulted in a 10% mortality. Similarly, the Middle East respiratory syndrome coronavirus (MERS-CoV) caused a pandemic in 2012 with a 37% mortality rate.

In late 2019 and early 2020, a novel coronavirus, SARS-coronavirus 2 (SARS-CoV-2), which is closely related to SARS-CoV, was discovered to be the cause of a large and rapidly spreading outbreak of respiratory disease, including pneumonia. Since the novel coronavirus was recognized, the disease it caused was termed coronavirus disease 2019 (CoVID-19).

The SARS-related coronaviruses are covered by spike proteins that contain a variable receptor-binding domain (RBD). This RBD binds to angiotensin-converting enzyme-2 (ACE-2) receptor found in the heart, lungs, kidneys, and gastrointestinal tract, thus facilitating viral entry into target cells.

There are four main genera within the subfamily Orthocoronavirinae, namely Alphacoronavirus (α-CoV), Betacoronavirus (β-CoV), Gammacoronavirus (γ-CoV) and Deltacoronavirus (δ-CoV). Both α- and β-coronavirus genera are known to infect mammals, whilst δ- and γ-CoVs infect birds. The S1 subunit of the betacoronavirus spike proteins displays a multidomain architecture, which shares little or no significant sequence similarity with the 51 subunit of alphacoronavirus spike proteins (Fang Li, J. Virol. 2012, 86(5):2856-2858). Two recent outbreaks of viral pneumonia caused by a 6-coronavirus are severe acute respiratory syndrome (SARS) and Middle East respiratory syndrome (MERS).

Influenza, commonly called “the flu”, is an infectious disease caused by influenza viruses. In each season (winter months in the Northern hemisphere) 5-15% of the population contracts influenza, with approx. 3-5 million severe cases. Over half a million deaths occur annually among high-risk groups, including young children, the elderly, and people with chronic health conditions. Following a 1-4 days incubation period, the onset of symptoms is sudden including fever, chills, headaches, muscle pain or aching, loss of appetite, fatigue, and confusion. Pneumonia may be caused by the primary viral infection or by a secondary bacterial infection, such as by the pneumococcus or S. aureus. Transmission of influenza is mediated by aerosol droplets. The primary infection site is the upper airways followed by progression to the lower airways and invasive infection.

There are four types (species) of influenza viruses: A, B, C and D. Seasonal epidemics (i.e. flu season) is caused by human influenza A (IAV) and B (IBV) viruses, while C and D are rarely associated with symptomatic infections in humans. Influenza A viruses are divided into subtypes based on two proteins on the surface of the virus: hemagglutinin (H) and neuraminidase (N). Although there are potentially 198 different influenza A subtype combinations, currently H1N1 and H3N2 types circulate worldwide. Influenza B viruses are not divided into subtypes, but instead are further classified into two lineages: B/Yamagata and B/Victoria. Based on these, seasonal flu vaccines usually contain two A (an H1N1 and an H3N2) and one or two B strains in combination. Influenza viruses have a negative-sense, single-stranded RNA genome that is segmented. Both IAV and IBV contain 8 segments, that could combine with the genome of other influenza viruses upon co-infection of the same cell. This process (called reassortment) give rise to progeny with significantly altered viral antigen composition. Such reassortant virus variants are novel to the human population and may give rise to pandemics (e.g. the Spanish flu in 1918, H1N1). Reassortment may also occur between viruses specific to different hosts. For instance, the “swine flu” pandemic in 2009 was caused by a triple reassortant virus carrying a combination of swine-, avian- and human-specific virus sequences (H1N1. Similarly, avian influenza strains, which are common in wild-water birds occasionally infect humans (“avian or bird flu”, H5N1). Upon reassortment of such avian viruses with human influenza viruses may give rise to variants enabling human-to-human spread, and therefore, have the potential to cause global pandemics.

Therapy of individuals of lower risk groups mainly focuses on symptomatic treatment (fever) and isolation. Patients suffering in severe or progressive clinical illness associated with suspected or confirmed influenza virus infection are treated with antiviral drugs (e.g. oseltamivir or other neuraminidase inhibitors) and supportive therapy (e.g. anti-inflammatory drugs). High risk groups (elderly, pregnant women, immunocompromised, associated chronic diseases) should receive annual seasonal flu vaccines as prophylaxis.

Adenoviruses are large non-enveloped viruses with a double-stranded DNA genome. The family Adenoviridae contains six genera with broad host specificity of viruses. All seven human adenovirus families (A to G) belong to the Mastadenovirus genus. Currently, the human viruses are classified into 88 different (sero)types, which are responsible to cause various mucosal infections. Types within families B (HAdV-B) and C (HAdV-C) are responsible for upper respiratory tract infections, HAdV-F (mainly types 40 and 41) and HAdV-G (mainly type 52) types cause gastroenteritis. Conjunctivitis is associated with HAdV-B and HAdV-D. These mucosal infections are usually self-limiting but can be more severe in an immunocompromised host. Adenovirus serotype 14 is an emerging pathogen that cause outbreaks of severe respiratory infections that could be lethal even in immunocompetent hosts.

Adenoviruses exhibit a classical icosahedral capsid made of 240 hexon and 12 penton proteins. The penton bases are associated with protruding fibers that bind to the host cell receptors: CD46 (family B) and coxsackie/adenovirus receptor (CAR, for all other families). This initial binding is followed by the interaction of the penton base viral structures with αV integrins, which stimulates endocytosis of the virus particles. Intracellularly, the capsid is destabilized, the endosome is degraded, and the viral DNA enters the nucleus through the nuclear pore. After associating with histone proteins the host transcription machinery is used for viral gene expression without the integration of the viral genome into the host genome. Viral proteins are expressed in an early (mainly regulatory proteins) and late (structural proteins) separated by the genome replication. Finally, the viral genomes are packaged into the protein shell and released from the host cell upon a virus induced lysis procedure.

Adenoviruses are relatively resistant to disinfectants and detergents (non-encapsulated), and survive long on surfaces and in water. There are no proven antiviral drugs to treat adenoviral infections, so treatment is largely directed at the symptoms. Currently, there is no adenovirus vaccine available to the general public, but a vaccine was used by the US military for types 4 and 7. Recently, engineered adenoviruses were successfully used in gene therapy and as viral vectors for heterologous vaccine antigen delivery (Ebola and Covid).

Human Respiratory Syncytial Virus (hRSV) is an enveloped virus with a negative-sense, single-stranded RNA genome. The genome is linear and has 10 genes encoding for 11 proteins. RSV is divided into two antigenic subtypes, A and B, with 16 and 22 clades (or strains), respectively.

RSV is highly contagious and can cause outbreaks from both community and hospital transmission. Each year, approximately 30 million acute respiratory illnesses and over 60,000 childhood deaths are caused RSV worldwide. Transmission occurs through contaminated aerosol droplets encountering mucosal surfaces of the nose, mouth, or eyes. Infection of the ciliated cells of the upper airways is followed by spread to the lower airways. It is among the most common childhood infections of various severity ranging from mild upper respiratory tract infection through bronchiolitis to viral pneumonia, which in the most severe cases may require mechanical ventilation. Immunocompromised individuals (including pre-term infants) have a higher risk for more severe diseases outcome. Therapeutic options are usually limited to supportive care, although ribavirin has been licensed for RSV infections in children. Vaccines are not available (despite significant development efforts), however, passive immunization with monoclonal antibodies (palivizumab) has become available as a choice for prophylaxis.

Calixarenes are macrocyclic compounds composed of phenolic units connected by methylene bridges to form a hydrophobic cylindrical shaped cavity that is capable of forming inclusion complexes with a variety of guest molecules. The wider side of the cavity is defined as the upper rim and the narrower hydroxyl side is the lower rim. The basic structure of a calixarene consisting of the repeating phenolic units is also understood as “calixarene backbone”. The phenolic units may be para-substituted phenols giving p-substituted calix[n]arene.

The upper rim or lower rim can be functionalized by chemical derivatization. Water soluble calixarenes can be made e.g. by sulfonation of the upper rim, thereby obtaining sulfocalixarenes, herein also referred to as sulfonatocalixarenes.

Calixarene applications are typically based on their biological properties and inclusion complex with drug.

Tsou et al. (Bioorg Med Chem Lett. 2010, 20(7): 2137-2139) discloses a dual inhibitor of HIV and HCBV infection based on a tetrabutoxy-calix[4]arene scaffold. A series of calix[4]arene derivatives was tested, such as containing a lower ring n-butyl group and an upper ring carboxamide-linked isophthalic group with a benzylester, free carboxylic acid and cyclohexylamide arm, respectively, on one of the carboxylic acids. A compound showing the most promising anti-HIV activity had no effect on the replication of herpes simplex virus-1 (HSV-1), HSV-2, and hepatitis B virus (HBV), but showed activity against hepatitis C virus (HCV). It was suggested that a calix[4]arene with four hydroxyl groups at the lower rim could stabilize the scaffold into a cone conformation through intramolecular hydrogen bonding. However, alkylation at the lower rim was essential for both anti-HIV and anti-HCV activities because locking the calix[4]arene scaffold into a cone conformation as the bulky substitutions are unable to invert through the ring.

Geller et al. (Antiviral Research 2010, 88(3):343-346) evaluated two calix[4]arene derivatives, the tetra-para-sulfonato-calix[4]arene (C[4]S) and the 1,3-bis(bithiazolyl)-tetra-para-sulfonato-calix[4]arene (C[4]S-BTZ) toward the human alpha coronavirus 229E (HCoV 229E), a common cold virus. Whereas (C[4]S) showed only very weak activity against HCoV 229E, (C[4]S-BTZ) was very effective and described as a promising virucidal (antiseptic) molecule.

### SUMMARY OF THE INVENTION

It is the objective of the present invention to provide new antiviral treatments, medicinal and pharmaceutical products which can be used to prevent from virus infection and/or virus spread, in particular in subjects that have been exposed to or infected with a virus, or who are at risk of being infected. The objective is solved by the subject of the present claims and as further described herein.

The invention provides for a calixarene compound in an antiviral effective amount for use as an anti-viral substance in a pharmaceutical preparation for use in prophylactic or therapeutic treatment of a disease condition which is caused by or associated with an infection with one or more viruses comprising at least one respiratory virus, or a co-infection or combination of at least one respiratory virus with one or more other viruses.

Specifically, said one or more other viruses are human pathogenic viruses, herein also referred to as human viruses.

Specifically, as subject is treated that is in need of an antiviral treatment targeting a respiratory virus, such as influenza virus, respiratory syncytial virus, adenovirus, coronavirus, or upper respiratory tract virus, such as rhinovirus.

A respiratory virus is herein specifically understood to be a virus causing respiratory disease. Though some of the target viruses further described herein may not only cause a respiratory disease, but also affect other body parts, such viruses are herein still understood to be a “respiratory virus”. Specifically, the antiviral treatment is targeting one or more human viruses, in particular a human respiratory virus, such as selected from the virus families Coronaviridae, Adenoviridae, Paramyxoviridae or Orthomyxoviridae.

Specifically, a disease condition is treated which is caused by or associated with an infection by one or more of:

a) Coronaviridae viruses, preferably selected from the group consisting of a β-coronavirus, such as SARS-CoV-2, MERS-CoV, SARS-CoV-1, HCoV-OC43, HCoV-HKU1, and an α-coronavirus, such as HCoV-NL63, HCoV-229E or PEDV, including naturally-occurring variants or mutants of any of the foregoing, or

b) Adenoviridae, preferably human Adenoviruses, such as HAdVB, HAdVC, or HAdVD;

c) human respiratory syncytial viruses (RSV), such as RSV subtype A or B; or

d) influenza viruses, such as human influenza viruses, preferably influenza virus A (IVA), such as H1N1, H3N3, or H5N1, or influenza virus B (IVB), or influenza virus C (IVC), or influenza virus D (IVD).

According to a specific aspect, said one or more viruses are selected from any of a) to d) above. Specifically, said respiratory virus is selected from any one of a) to d) above.

According to a specific aspect, said one or more viruses comprise any one or both of SARS-CoV-2 and influenza virus.

Specifically, said disease condition is caused by co-infection with said one or more viruses.

Specifically, the calixarene compound is used for prophylactic treatment to prevent from virus infection, spread and/or disease, by administering to a subject, in particular by application to exposed biological surfaces of the subject.

According to a specific aspect, the pharmaceutical preparation is a medicinal product or a drug product. Specifically, the pharmaceutical preparation comprises the calixarene compound and a pharmaceutically acceptable carrier.

Specifically, a disease condition is treated which is caused by or associated with an infection by one or more Coronaviridae viruses, in particular one or more different Coronaviridae viruses.

According to a specific aspect, said one or more viruses comprise a coronavirus.

Specifically, the coronavirus is a human coronavirus, in particular a virus that effects human beings.

Specifically, the coronavirus is a naturally-occurring virus, which is identified as a coronavirus, including naturally-occurring mutants of any such coronavirus.

Specifically, the coronavirus is a β-coronavirus, or naturally-occurring mutants thereof.

According to a specific aspect, the coronavirus is not a coronavirus that causes common cold, such as HCoV 229E.

Specifically, the coronavirus is a β-coronavirus, more specifically a human β-coronavirus. Within the genus β-coronavirus, four lineages (i.e., A, B, C, and D) are commonly recognized. Specifically, the β-coronavirus is of the B-lineage (subgenus Sarbecovirus), such as SARS-CoV or SARS-CoV-2, or of the C-lineage (subgenus Merbecovirus), such as MERS-CoV, or of the A-lineage (subgenus Embecovirus), such as HCoV-OC43 or HCoV-HKU1.

Specifically, the β-coronavirus is selected from the group consisting of SARS-CoV-2, MERS-CoV, SARS-CoV-1, HCoV-OC43, and HCoV-HKU1, or mutants thereof, in particular those mutants which are naturally-occurring, i.e which are not artificial (“man-made”) but found in nature.

Specifically, said one or more different Coronaviridae viruses referred to herein are naturally-occurring SARS-CoV-2 variants or mutants, in particular those that comprise one or more mutations of the spike protein (SARS-CoV-2 S-protein), such as any one or more of the following mutations: K417N, L452R, N501Y, D614G, P681H, P681R, E484K, E484Q, or 69/70 deletion.

Specifically, the spike protein comprises or consists of the amino acid sequence identified as SEQ ID NO:1 (sequence provided in FIG. 5, NCBI accession number QII57161.1, SARS-CoV-2, S-protein).

Preferably said one or more different Coronaviridae viruses are naturally-occurring SARS-CoV-2 variants or mutants selected from the group consisting of the UK (B1.1.7) variant, the South African (B.1.351) variant, the Brazilian (P.1) variant, the Indian (B.1.617) variant and the Bengal (B.1.618) variant.

According to a specific aspect, the pharmaceutical preparation is a medicinal product or a drug product, comprising the calixarene compound and a pharmaceutically acceptable carrier.

According to a specific aspect, the disease condition is common cold, infection of the nose, sinusitis, throat and larynx, bronchiolitis, diarrhea, rash on skin, or pneumonia, acute respiratory distress syndrome (ARDS). Specifically, a disease condition can be a symptom associated with any one or more of the foregoing. Specifically, a symptom to be treated can be any of coughing, sore throat, runny nose, sneezing, headache, and fever. Specifically, the disease condition is associated with or caused by a target virus, or a respiratory virus described herein, in particular wherein the target virus is a virus of the Coronaviridae, Adenoviridae, Paramyxoviridae, or Orthomyxoviridae family.

According to a specific aspect, the antiviral effective amount is effective in preventing infection of susceptible cells by the virus, thereby treating the disease condition. Specifically, susceptible cells are within or at a biological surface or a subject.

According to a specific aspect, the antiviral effective amount is 0.1-500 μg/dose. preferably below any one of 100, 90, 80, 70, 50, 50, 40, 30, 20, 10, 9, 8, 7, 6, 5, 4, 3, 2, or 1 μg/dose. Specifically, the number of doses is up to 1 to 10 per day.

Specifically, the antiviral effective amount may be an amount ranging amount ranging 15 mg-150 mg, or even less, such as less than any one of 50, 40, 30, 20, 10, 9, 8, 7, 6, 5, 4, 3, 2, 1, 0.9, 0.8, 0.7, 0.6, 0.5, 0.4, 0.3, 0.2, or 0.1% of the amount ranging 15 mg-150 mg. Such lower amounts have particularly been proven to be effective with coronaviruses (or viruses of the family Coronaviridae), and/or viruses other than coronaviruses (or other than viruses of the family Coronaviridae), such as viruses of the families Adenoviridae, Paramyxoviridae, or Orthomyxoviridae.

Specifically, the calixarene compound comprises a calixarene backbone composed of a number of substituted or unsubstituted phenolic units connected by methylene bridges. Specifically, the number of phenolic units is 4, 5, 6, 7, or 8, preferably 4, 6, or 8. Specifically, the calixarene compound comprises a number of phenolic unit repeats. Specifically, the phenolic units may be para-substituted phenols giving p-substituted calix[n]arene. Specifically, the calixarene compound comprises hydroxyl groups on each phenolic unit at the lower rim.

According to a specific aspect, the calixarene compound is a water soluble calixarene. Specifically, water soluble calixarenes are sulfonated or carboxylated at the upper rim, thereby obtaining sulfonatocalixarenes, also referred to as sulfocalixarenes.

Specifically, the calixarene compound does not comprise hydrophobic or non-polar groups in the para-substituted phenols and/or at the upper rim.

According to a specific aspect, the calixarene compound does not comprise a drug substance or an active substance, such as antiviral substances. Specifically, the calixarene compound does not comprise a drug conjugate. Specifically, the calixarene compound does not comprise an inclusion complex with a drug, and/or does not carry a drug.

According to a specific aspect, the calixarene compound is a calix[n]arene characterized by the structure (I):

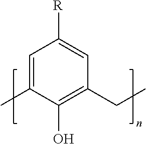

wherein n=denotes 4, 5, 6, 7, or 8 and

R is selected from the group consisting of —SO₃R^{a}, —OR^{a}, —COOR^{a}, hydrogen, or halogen, and

each R^{a} is hydrogen or methyl;

or a pharmaceutically acceptable salt thereof.

Specifically, the calix[n]arene is a calix[4]arene with four intrannular hydroxyl groups on the lower rim and four sulfonate or carboxilate groups on the upper rim, preferably wherein the calixarene compound is Sulfonatocalixarene, or a physiologically acceptable salt thereof, such as Sulfonatocalixarene sodium salt.

Specifically, the calixarene compound is p-sulfonatocalix[4]arene, or 5,11,17,23-sulfonato-25,26,27,28-tetrahydroxycalix[4]arene (herein also referred to as “CX-1”), or a physiologically acceptable salt thereof, such as a sodium salt of 5,11,17,23-sulfonato-25,26,27,28-tetrahydroxycalix[4]arene.

4-Sulfocalix[4]arene, structure (I)

CAS Number 112269-92-8

Empirical Formula (Hill Notation) C₂₈H₂₄O₁₆S₄

Molecular Weight 744.74

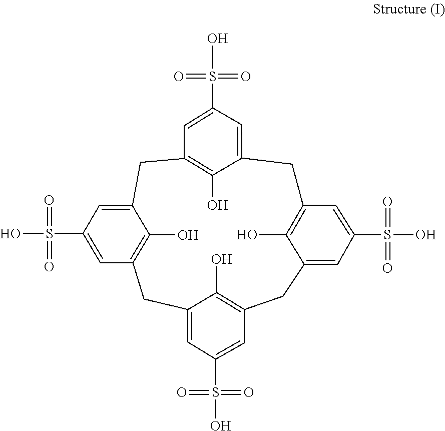 [SMILES: O=S(=O)(O)c1cc2c(O)c(c1)Cc1cc(S(=O)(=O)O)cc(c1O)Cc1cc(S(=O)(=O)O)cc(c1O)Cc1cc(S(=O)(=O)O)cc(c1O)C2]

4-Sulfocalix[4]arene sodium salt, structure (II)

CAS Number 151657-13-5

Linear Formula C₂₈H₂₄₋ₓO₁₆S₄Naₓ

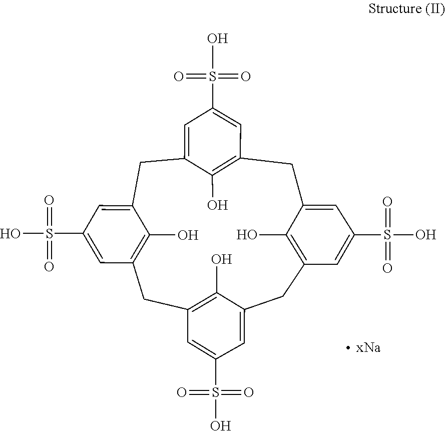 [SMILES: O=S(=O)(O)c1cc2c(O)c(c1)Cc1cc(S(=O)(=O)O)cc(c1O)Cc1cc(S(=O)(=O)O)cc(c1O)Cc1cc(S(=O)(=O)O)cc(c1O)C2]

The preparations, methods and uses described herein are specifically comprising the calixarene compound in an antiviral effective amount. Such antiviral effective amount may target one specific virus or more than one different viruses, virus mutants or virus variants, such as further described herein.

The target virus of an antiviral effect is particularly understood as the virus which is (directly or indirectly) related to the indication for the treatment described herein, in particular the treatment for prophylaxis or therapy of a disease condition or disease that is caused or otherwise associated by such target virus.

Specifically, the target of the preparations, methods and uses described herein, and in particular the target of an antiviral effect described herein, can be any one or more different virus species, virus mutants or virus variants e.g., 1, 2, 3, 4, 5, 6, 7, 8, 9, or 10 different virus species, mutants or variants.

Specifically, the target can be any two or more viruses e.g., 2, 3, 4, 5, 6, 7, 8, 9, or 10 different virus species, virus mutants or virus variants, wherein at least 2, 3, or 4 of the viruses are of different virus families, such as selected from the families:

a) Coronaviridae (β-coronavirus, such as SARS-CoV-2, MERS-CoV, SARS-CoV-1, HCoV-OC43, HCoV-HKU1; or α-coronavirus, such as HCoV-NL63, HCoV-229E or PEDV, including naturally-occurring variants or mutants of any of the foregoing);

b) Adenoviridae (such as Adenoviruses or human Adenoviruses e.g., HAdVB, HAdVC, or HAdVD);

c) Paramyxoviridae (such as RSV or human RSV e.g., hRSV subtype A or B); or

d) Orthomyxoviridae (such as influenza viruses or human influenza viruses, preferably influenza virus A (IVA), such as H1N1, H3N3, or H5N1, or influenza virus B (IVB), or influenza virus C (IVC), or influenza virus D (IVD)).

Specifically, such viruses listed in a) to d) above are herein referred to as exemplary “respiratory viruses”.

According to a specific example, the target can be at least one virus selected from a) and at least one virus selected from b), c), or d). Specifically, the target can be at least one β-coronavirus, such as a SARS virus, in particular SARS-CoV-2, and at least one influenza virus.

Specifically, the target can be at least one β-coronavirus, such as a SARS virus, in particular SARS-CoV-2, and at least one Adenovirus HAdVB, HAdVC, or HAdVD.

Specifically, the target can be at least one β-coronavirus, such as a SARS virus, in particular SARS-CoV-2, and at least one hRSV subtype A or B.

According to a specific example, the target can be at least one virus selected from d) and at least one virus selected from a), b), or c).

Specifically, the target can be at least one influenza viruses or human influenza viruses, and at least one Adenovirus HAdVB, HAdVC, or HAdVD.

Specifically, the target can be at least one influenza viruses or human influenza viruses, and at least one hRSV subtype A or B.

According to a specific example, the target can be at least one virus selected from b) and at least one virus selected from a), c), or d).

Specifically, the target can be at least one Adenovirus HAdVB, HAdVC, or HAdVD, and at least one hRSV subtype A or B.

According to a specific example, the target can be at least one virus selected from a) and at least one virus selected from b), and optionally at least one virus selected from c), or d). Specifically, the target can be at least one β-coronavirus, such as a SARS virus, in particular SARS-CoV-2, and at least one influenza virus, and optionally at least one one Adenovirus HAdVB, HAdVC, or HAdVD, and/or at least one hRSV subtype A or B.

According to a specific aspect, the antiviral effective amount is administered locally or systemically.

According to a specific aspect, said pharmaceutical preparation is formulated for local administration, such as for topical or topical mucosal administration, preferably for application to the upper and lower respiratory tract, nasal, pulmonary, intraoral, ocular, or dermal use, or for systemic administration, preferably by intravenous, intramuscular, subcutaneous, intradermal, transdermal, or oral administration. Typically, for parenteral administration, intravenous or peroral administration is preferred.

According to a specific aspect, said pharmaceutical preparation is administered to a subject as a spray, such as a nose spray, a powder, such as an instant powder or powder for inhalation, or by a healthcare device, such as e.g., comprising a surface or fabric impregnated with the calixarene compound for inhalation, a gel, an ointment, a cream, a foam, or a liquid solution, a lotion, a gargle solution, an aerosolized powder, an aerosolized liquid formulation, granules, capsules, drops, tablet, syrup, lozenge, or a preparation for infusion or injection.

Specifically, antiviral formulations and administration forms are provided, such as for veterinary and for human use. Specifically, the formulations comprise a predetermined amount of the calixarene compound as active ingredient; e.g., as a powder or granules; as a solution or a suspension in an aqueous or non-aqueous liquid; or as an oil-in-water liquid emulsion or a water-in-oil liquid emulsion.

According to a specific aspect, the calixarene compound is used in an antiviral effective amount to provide a peak concentration (or maximum concentration) in blood or plasma, which is within the range of about 0.01-5 mg/mL or about 0.01-15 mg/mL, and/or up to any one of 15, 10, 9, 8, 7, 6, 5, 4, 3, 2, 1, 0.9, 0.8, 0.7, 0.6, 0.5, 0.4, 0.3, 0.2, or 0.1 mg/mL.

According to a specific aspect, the calixarene compound is used in a formulation at a concentration of at least any one of 0.001, 0.01, 0.05, 0.1, 0.2, 0.3, 0.4, 0.5, 1, 1.5 or 2 mM, and/or up to any one of 10, 9, 8, 7, 6, 5, 4, 3, 2, 1, 0.9, 0.8, 0.7, 0.6, 0.5, 0.4, 0.3, 0.2, 0.1, 0.09, 0.08, 0.07, 0.06, 0.05 mM, preferably within the range of 1 μM-10 mM.

Specifically, a liquid solution or dispersion is used for local administration, such as nasal administration, e.g., by nose drops or a nasal spray, preferably wherein the antiviral effective amount is at least any one of 0.002, 0.003, 0.004, 0.005, 0.01, 0.02, 0.03, 0.04, 0.05, 0.06, 0.07, or 0.08 mg per nostril, and/or up to any one of 2.5, 2, 1.5, 1, 0.9, 0.8, 0.7, 0.6, 0.5, 0.4, 0.3, 0.2, 0.1, or 0.05 mg per nostril, preferably within the range of 0.01-2.5 mg or 0.005-2.5 mg per nostril.

Specifically, a dose can be administered by local administration, in particular nasal administration which is at least any one of 0.002, 0.003, 0.004, 0.005, 0.01, 0.02, 0.03, 0.04, 0.05, 0.06, 0.07, 0.08, 0.09, 1.0, 1.1, 1.2, 1.3, 1.4, 1.5, or 1.6 mg per dose, and/or up to any one of 5, 4, 3, 2, 1, 0.9, 0.8, 0.7, 0.6, 0.5, 0.4, 0.3, 0.2, or 0.1 mg per dose, preferably within the range of 0.05-5 mg or 0.025-5 mg per dose.

A formulation is preferably applied as a nasal spray, nasal drops, an aerosol such as an aerosolized liquid or powder e.g., as a throat spray or for intrapulmonary administration.

Exemplary formulations may contain the calixarene compound as an active ingredient in an amount of, for example, 0.001 to 2% (w/w), such as at least about any one of 0.001%, 0.002%, 0.003%, 0.004%, 0.005%, 0.006%, 0.007%, 0.008%, 0.009%, 0.01%, 0.02%, 0.03, 0.04, 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, and/or up to 2.0% (w/w), preferably within the range of 0.05 to 2% (w/w).

Specifically, a volume of 100-1000 μL per dose can be applied in a sprayable formulation, e.g., up to 500 μL spray volume. According to a specific example, a nasal spray may deliver a volume of about 100-150 μL per spray. Typically, two sprays are applied per nostril one or twice daily.

When using a spray, a metered spray is preferably used to administer a certain spray volume or dose per puff. Specifically, the calixarene compound is formulated as a metered-spray solution for intranasal administration. A nasal spray may contain 0.1% or 0.15% of the calixarene compound USP in an aqueous solution at pH 6.8±0.3.

Formulations suitable for intrapulmonary administration may have a particle size in the range of 0.1 to 500 microns, which can be administered by inhalation through the nasal passage or by inhalation through the mouth so as to reach the alveolar sacs. Suitable formulations include aqueous or oily solutions of the calixarene compound. Formulations suitable for aerosol or dry powder administration may be prepared according to conventional methods and may be delivered with other therapeutic agents such as compounds used in the treatment or prophylaxis of lung inflammation or lung diseases.

Specifically, a formulation is used for systemic, in particular parenteral administration.

Specifically, a liquid solution or dispersion is used for parenteral administration, such as by infusions or injections, preferably wherein the antiviral effective amount provides a dose of about at least any one of 0.5, 1, 1.5, 2, 2.5, 3, 3.5, 4, 4.5, 5, 5.5, 6, 6.5, 7, 7.5, 8, 8.5, 9, 9.5 or 10 g per dose, preferably within the range of 0.5 to 10 g per dose.

Specifically, a single loading dose followed by maintenance doses, e.g., by daily administration for 1-10 days, or until a certain clinical response has been achieved. The loading dose is typically higher than the maintenance dose.

According to a specific aspect, an antiviral effective systemic dose is at least about any one of 30, 40, 50, 60, 70, 80 or 90 mg/kg and/or up to about any one of 100, 90, 80, 70, 60, 50, 40, or 30, preferably within the range of 30-100 mg/kg.

Specifically, a tablet, gel or lozenge is used for oral administration, preferably wherein the antiviral effective amount is at least any one of 0.1, 0.5, 1, 1.5, 2, 2.5, 3, 3.5, 4, 4.5, 85 g, 6 g, 7 g, or 8 g per daily dose, and/or up to any one of 8, 7, 6, 5, 4.5, 4, 3.5, 3, 2.5, 2, 1.5, 1, 0.5, or 0.1 g per daily dose, preferably within the range of 0.1 to 8 g, preferably about 4.5 g per daily dose.

Specifically, a tablet comprising the calixarene compound may be used which can be administered once to three times a day.

Formulations suitable for topical administration in the mouth include lozenges comprising the active ingredient in a flavored basis, such as sucrose, acacia or tragacanth, pastilles comprising the calixarene compound in an inert basis such as gelatin and glycerin, sucrose, or acacia, or mouthwashes comprising the calixarene compound in a suitable liquid carrier.

When formulated in a topically applied gel or ointment, the active ingredients may be employed with either a paraffinic or a water-miscible ointment base. Alternatively, active ingredients may be formulated in a cream with an oil-in-water cream base.

Formulations suitable for topical administration to the eye may include eye drops, gel or cream, wherein the calixarene compound is dissolved or suspended in a suitable carrier, especially an aqueous solvent or oil/water emulsion.

According to specific examples, such formulations for a topical administration in the mouth, or a topically applied formulation such as a gel or ointment, may comprise the calixarene compound in a concentration of at least about any one of 0.05%, 0.06%, 0.07%, 0.08%, 0.09%, 0.1%, 0.15%, 0.2%, 0.3%, 0.4%, 0.5%, 0.6%, 0.7%, 0.8%, 0.9%, 1.0%, 2%, 3%, 4%, 5%, 6%, 7%, 8%, 9%, 10%, 11%, 12%, 13%, 14%, 15%, 16%, 17%, 18%, 19%, and/or up to 20% (w/w), preferably within the range of 0.05 to 20% (w/w).

According to a specific aspect, treatment with the calixarene compound can be combined with a further treatment administering a calixarene compound (which can be the same or a different compound) in another administration form. For example, treatment with an intranasal or throat spray comprising a Sulfocalixarene sodium salt can be combined with a tablet comprising a calixarene compound (which can be a Sulfocalixarene sodium salt or different from such Sulfocalixarene sodium salt).

According to a specific aspect, the calixarene compound is administered as the sole antiviral substance, or wherein treatment is combined with a further treatment such as an additional antiviral, anti-inflammatory and/or antibiotic treatment e.g., comprising administration of one or more antiviral substances or agents, and/or one or more anti-inflammatory and/or antibiotic substances or agents, by one or more different preparations and/or one or more different routes of administration.

Specifically, the calixarene compound can be combined with one or more additional active therapeutic agents in a unitary dosage form for simultaneous, co-administration or sequential administration to a subject. The combination therapy may e.g., be administered as a simultaneous, parallel or sequential regimen. When administered sequentially, the combination may be administered in two or more administrations.

According to a specific aspect, a subject is treated who has been infected or is at risk of being infected with said virus, preferably a human being, or a non-human mammal, such as a dog, cat, horse, camelids, cattle or pig.

Specifically, the subject is or has been exposed to a virus, or is otherwise at risk of being infected with the virus.

Specifically, the subject has been determined or diagnosed of being infected with the virus.

In specific embodiments, a subject is treated which is a diseased subject or patient or patient at risk of suffering from a target virus or Coronaviridae virus-caused disease, such as gastroenteritis, respiratory tract disease, or severe acute respiratory syndrome (SARS). Specifically, the disease is a β-coronavirus-caused disease e.g., a SARS virus-caused disease, upon getting in contact with the pathogen, such as COVID19, or COVID19-associated pneumonia.

In a further specific embodiment, a subject is treated which is a diseased subject or patient at risk of or suffering from influenza-virus caused disease, such as influenza, or a RSV virus caused disease or adenovirus caused disease

The invention further provides for the calixarene compound as described herein for use as an antiviral substance in a medicinal product for treating a biological surface to prevent from virus infection and/or virus spread.

According to a specific aspect, said medicinal product is formulated for topical use, preferably for application to the upper and lower respiratory tract, nasal, pulmonary, intraoral, ocular, or dermal use.

Topical application typically refers to the surface of the skin, a wound, and/or mucosal cells or tissues (e.g., alveolar, buccal, lingual, masticatory, or nasal mucosa, etc.).

According to a specific aspect, said medicinal product is used in a formulation suitably used for topical administration, in particular mucosal administration, such as a spray, solution, dispersion, dry powder, or aerosolized liquid or powder.

According to a specific aspect, the calixarene compound is applied to a biological surface in an antiviral effective amount, preferably wherein the amount is 1 ng-1000 ng per cm², preferably 10-800 ng/cm², or up to any one of 800, 700, 600, 500, 400, 300, 200, 100, 90, 80, 70, 60, 50 ng/cm².

Any of the medicinal products suitably used for topical treatment as described herein can be used for treating the biological surface.

According to a specific aspect, the biological surface comprises or consists of a mucosal surface which is infected or at risk of being infected with said virus.

The invention further provides for the use of a calixarene compound as described herein, as viral disinfectant, in particular suitable for treating a biological surface, or a non-biological surface, such as sanitary devices face masks, etc. Specifically, animate or non-animate surfaces can be treated using the disinfectant.

Specifically, the viral disinfectant is an antiviral preparation, such as a medicinal product.

According to specific aspect, the invention provides for a calixarene compound as described herein, for use in preventing or treating a coronavirus infection, in particular a SARS virus infection and/or an influenza infection, in a human or non-human mammal.

According to a specific aspect, a kit is provided which comprising one or more individual dosage units of the calixarene compound as further described herein, and directions for their use in treating a coronavirus infection or a coronavirus-caused disease, in a human or non-human mammal.

According to a specific aspect, the invention provides for an antiviral pharmaceutical preparation comprising the calixarene compound as further described herein and a pharmaceutically acceptable carrier.

Specifically, the pharmaceutical preparation is provided for medical use, in particular, for use in the prophylactic or therapeutic treatment of a disease condition caused by a coronavirus such as COVID19.

Specifically, the pharmaceutical preparation is provided for medical use, in particular, for use in the prophylactic or therapeutic treatment of a disease condition caused by a Coronaviridae virus and/or an Adenoviridae virus and/or a Paramyxoviridae virus and/or a Orthomyxoviridae virus.

According to a specific aspect, the invention further provides for methods of treating a subject being infected or at risk of being infected with one or more different viruses such as one or more of Coronaviridae viruses and/or Adenoviridae viruses and/or Paramyxoviridae viruses and/or Orthomyxoviridae viruses, comprising administering an antiviral effective amount of the calixarene compound, and respective medicinal products or pharmaceutical preparations as further described herein.

According to a specific aspect, the invention further provides for methods of treating a subject being infected or at risk of being infected with a target virus such as described herein, e.g., a coronavirus, comprising administering an antiviral effective amount of the calixarene compound, and respective medicinal products or pharmaceutical preparations as further described herein.

According to specific aspect, the invention provides for a calixarene compound as described herein, for use in preventing or treating a Coronaviridae viral infection, in particular a SARS virus infection, in a human or non-human mammal.

According to specific aspect, the invention provides for a calixarene compound as described herein, for use in preventing or treating an Adenoviridae viral infection, in particular a HAdVB, HAdVC, or HAdVD virus infection in a human being.

According to specific aspect, the invention provides for a calixarene compound as described herein, for use in preventing or treating a Paramyxoviridae viral infection, in particular a human RSV virus infection, in a human being.

According to specific aspect, the invention provides for a calixarene compound as described herein, for use in preventing or treating a Orthomyxoviridae viral infection, in particular an influenza virus infection, in a human or non-human mammal.

According to a specific aspect, a kit is provided which comprising one or more individual dosage units of the calixarene compound as further described herein, and directions for their use in treating a Coronaviridae viral infection or a Coronaviridae virus-caused disease, in a human or non-human mammal.

According to another specific aspect, a kit is provided which comprising one or more individual dosage units of the calixarene compound as further described herein, and directions for their use in treating an Adenoviridae viral infection, in particular a HAdVB, HAdVC, or HAdVD virus infection in a human being.

According to another specific aspect, a kit is provided which comprising one or more individual dosage units of the calixarene compound as further described herein, and directions for their use in treating a Paramyxoviridae viral infection, in particular a human RSV virus infection, in a human being.

According to another specific aspect, a kit is provided which comprising one or more individual dosage units of the calixarene compound as further described herein, and directions for their use in treating a Orthomyxoviridae viral infection, in particular an influenza virus infection, in a human or non-human mammal.

According to a further specific aspect, the invention provides for an antiviral preparation of the calixarene compound as described herein (such as a medicinal product, pharmaceutical preparation or disinfectant) and methods of producing such antiviral preparation comprising formulating an antiviral effective amount of the calixarene compound with a pharmaceutically acceptable carrier to produce an antiviral preparation, in particular a medicinal product or pharmaceutical preparation.

The topical administration of any of the antiviral preparations as described herein (such as a medicinal product, pharmaceutical preparation or disinfectant) to a biological surface is preferably such that upon a certain contact time of e.g. 10 minutes to 24 hours, and/or up to 24, 18, 12, 6, 5, 4, 3, 2, or 1 hour(s), contacting results in at least a 0.5-fold (by half) reduction, or 1-log, 2-log, 3-log, 4-log, 5-log reduction in a virus on said surface.

### FIGURES

FIG. 1 shows microscopic pictures of Vero E6 cells in an in vitro model of SARS-CoV-2 infection when cells were infected in the presence of CX-1. A: Negative control: uninfected cells; B: Positive control: cells infected with SARS-CoV-2; C-E: Cells infected with SARS-CoV-2 in the presence of CX-1 used at 500, 100 or 20 μM concentrations, respectively. Cells were infected with MOI 0.01 SARS-Co-V2 virus, and microscopic pictures taken 48 hours post infection.

FIG. 2 shows microscopic pictures of Vero E6 cells in an in vitro model of SARS-CoV-2 infection when cells were infected first with virus then treated with CX-1. A: Negative control: uninfected cells; B: Positive control: cells infected with SARS-CoV-2; C-E: Cells infected with SARS-CoV-2 in the presence of CX-1 used at 500, 100 or 20 μM concentrations, respectively. Cells were infected with MOI 0.01 SARS-CoV-2 virus for 30 min, then culture medium was replaced to remove the virus and cell were cultured in the presence of CX-1, microscopic pictures taken 48 hours post infection.

FIG. 3 shows the antiviral effect of CX-1 in a prophylactic setting. Vero E6 cells were infected with SARS-CoV-2 in the presence of CX-1 at the indicated concentrations. After 30 min of incubation at 37° C., the virus-containing medium was replaced with fresh medium supplemented with the corresponding concentrations of CX-1. Following an additional 48 h incubation at 37° C., viral copy numbers were determined by qPCR.

FIG. 4 shows the influence of CX-1 as well as oseltamivir on the viral copy numbers obtained from apical washes of reconstituted nasal tissues at 24 h (panel A) or 48 h (panel B) after infection by influenza virus H1N1. Differences between two groups were tested by Student's t test using GraphPad Prism 6.0. Statistically significant differences are marked by asterisks. Graphs shows mean results ±standard error of mean of triplicates from one representative experiment. *P<0.05

FIG. 5 shows the effect of CX-1 as well as oseltamivir on the cilia beating frequency of primary human nasal epithelial cells at day 4 post-infection by influenza virus H1N1. Differences between two groups were tested by Student's t test using GraphPad Prism 6.0 Statistically significant differences are marked by asterisks.

Graphs shows mean±standard error of mean of triplicates from one representative experiment. ***P<0.001; ****P<0.0001.

FIG. 6: SEQ ID NO:1 (NCBI accession number QII57161.1, SARS-CoV-2, S-protein).

### DETAILED DESCRIPTION OF THE INVENTION

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−10% or +/−5% of the given value.

A small molecule organic compound such as a calixarene compound as described herein may be used as a “physiologically acceptable salt”. The choice of salt is determined primarily by how acid or basic the chemical is (the pH), the safety of the ionized form, the intended use of the drug, how the drug is given (for example, by mouth, injection, or on the skin), and the type of dosage form (such as tablet, capsule, or liquid).

Exemplary salts which are physiologically acceptable are sodium salts. However, it is, also possible to employ, in place of the sodium salts, other physiologically acceptable salts, e.g., other alkali metal salts, alkaline earth metal salts, ammonium salts and substituted ammonium salts. Specific examples are the potassium, lithium, calcium, aluminum and iron salts. Preferred substituted ammonium salts are those derived, for example, from lower mono-, di-, or trialkylamines, or mono-, di- and trialkanolamines. The free amino acids per se can also be used. Specific examples are ethylamine, ethylenediamine, diethylamine, or triethylamine salts.

The term “pharmaceutically acceptable” also referred to as “pharmacologically acceptable” means compatible with the treatment of animals, in particular, humans. The term pharmacologically acceptable salt also includes both pharmacologically acceptable acid addition salts and pharmacologically acceptable basic addition salts.

The term “pharmacologically acceptable acid addition salt” as used herein means any non-toxic organic or inorganic salt of any base compound of the disclosure, or any of its intermediates. Basic compounds of the disclosure that may form an acid addition salt include, for example, compounds that contain a basic nitrogen atom. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono-, di- or the triacid salts can be formed, and such salts may exist in either a hydrated, solvated or substantially anhydrous form. In general, the acid addition salts of the compounds of the disclosure are more soluble in water and various hydrophilic organic solvents, and generally demonstrate higher melting points in comparison to their free base forms. The selection of the appropriate salt will be known to one skilled in the art. Other non-pharmacologically acceptable acid addition salts, e.g. oxalates, may be used, for example, in the isolation of the compounds of the disclosure, for laboratory use, or for subsequent conversion to a pharmacologically acceptable acid addition salt.

The term “pharmacologically acceptable basic salt” as used herein means any non-toxic organic or inorganic basic addition salt of any acid compound of the invention, or any of its intermediates, which are suitable for or compatible with the treatment of animals, in particular humans. Acidic compounds of the invention that may form a basic addition salt include, for example compounds that contain carboxylic acid, sulfonic acid, sulfinic acid, sulfonamide, N-unsubstituted tetrazole, phosphoric acid ester, or sulfuric acid ester. Illustrative inorganic bases which form suitable salts include lithium, sodium, potassium, calcium, magnesium, or barium hydroxide. Illustrative organic bases which form suitable salts include aliphatic, alicyclic or aromatic organic amines such as methylamine, trimethylamine and picoline or ammonia. The selection of the appropriate salt will be known to a person skilled in the art. Other non-pharmacologically acceptable basic addition salts, may be used, for example, in the isolation of the compounds of the invention, for laboratory use, or for subsequent conversion to a pharmacologically acceptable basic addition salt. The formation of a desired compound salt is achieved using standard techniques. For example, the neutral compound is treated with a base in a suitable solvent and the formed salt is isolated by filtration, extraction or any other suitable method.

The term “antiviral” as used herein shall refer to any substance, drug or preparation, that effects the biology of a virus and attenuates or inhibits viral attachment, entry, replication, shedding, latency or a combination thereof, resulting in reduction of viral load or infectivity. The terms “attenuating,” “inhibiting,” “reducing,” or “prevention,” or any variation of these terms, when used in the claims and/or the specification includes any measurable decrease or complete inhibition to achieve a desired result, e.g., reduction in the risk of viral infection (pre-exposure), or reduction of post-exposure viral survival, load, or growth.

Exemplary antiviral preparations described herein are medicinal products, pharmaceutical preparations, or disinfectants, for in vivo, ex vivo or in vitro use.

The term “biological surface” as used herein shall refer to a surface comprising viable cells, such as mammalian (human or non-human animal) cells, including e.g., a biological tissue surface, such as a surface or epithelial or dermal tissue (e.g. skin), mucosal tissue, or membrane tissue.

The term “effective amount” with respect to an antiviral effect as used herein, shall refer to an amount (in particular a predetermined amount) that has a proven antiviral effect. The amount is typically a quantity or activity sufficient to, when applied to a surface or administered to a subject effect beneficial of desired results, including antiviral or clinical results, and, as such, an effective amount or synonym thereof depends upon the context in which it is being applied.

An effective amount of a pharmaceutical preparation or drug is intended to mean that amount of a compound that is sufficient to treat, prevent or inhibit a disease, disease condition or disorder. Such an effective dose specifically refers to that amount of the compound sufficient to result in healing, prevention or amelioration of conditions related to diseases or disorders described herein.

In the context of disease, effective amounts (in particular prophylactically or therapeutically effective amounts) of a calixarene compound as described herein are specifically used to treat, modulate, attenuate, reverse, or affect a disease or condition that benefits from its antiviral effect. The amount of the compound that will correspond to such an effective amount will vary depending on various factors, such as the given drug or compound, the formulation, the route of administration, the type of disease or disorder, the identity of the subject or host being treated, the assessment of the medical situations and other relevant factors, but can nevertheless be routinely determined by one skilled in the art.

A treatment or prevention regime of a subject with an effective amount of the calixarene compound described herein may consist of a single application or administration, or alternatively comprise a series of applications and administrations, respectively. For example, the calixarene compound may be used at least once a month, or at least once a week, or at least once a day. However, in certain cases of an acute phase, e.g. upon suspected or confirmed exposure to a virus, or after virus infection has been determined, the calixarene compound may be used more frequently e.g., 1-10 times a day.

Specifically, a combination therapy is provided which includes treatment with the preparation described herein and standard therapy of a coronavirus-caused disease.

Doses may be applied in combination with other active agents such as antiviral agents, anti-inflammatory drugs or antibiotics, e.g. upon the subject's risk of viral spread, so to prevent a pathogen associated reaction.

Treatment can be combined with an antiviral, anti-inflammatory or antibiotic treatment, preferably wherein a pharmaceutical preparation is administered before, during (e.g., by co-administration or in parallel), or after said antiviral, anti-inflammatory or antibiotic treatment.

Specifically, the calixarene compound described herein can be combined with an additional antiviral agent, which can be a calixarene compound, e.g. the same of a different compound. Specific embodiments refer to further antiviral agents selected from an ACE2 inhibitor, a viral protein M2 ion channel inhibitor, a neuraminidase inhibitor, an RNA replication and translation inhibitor and a polymerase inhibitor. The antiviral agent may be amantadine or rimantadine. Specifically, the antiviral agent may be oseltamivir, zanamivir, peramivir, ribavirin, lopinavir, or ritonavir. Specific further antiviral examples are those suitably used for biological surface treatment such as carrageenan, or those currently under investigation for treating SARS-Cov2 infections, such as hydroxychloroquine, or remdesivir.

Specifically, the calixarene compound is combined with an anti-inflammatory agent such as standard steroidal anti-inflammatory drugs, glucocorticoids and nonsteroidal anti-inflammatory drugs (NSAID's). Suitable NSAID's include, but are not limited to ibuprofen, naproxen, fenoprofen, ketoprofen, flurbiprofen, oxaprozin, indomethacin, sulindac, etodolac, ketorolac, diclofenac, nabumetone, piroxicam, meloxicam, tenoxicam, droxicam, lornoxicam, isoxicam, mefenamic acid, meclofenamic acid, flufenamic acid, tolfenamic acid and celecoxib. Suitable steroidal anti-inflammatory agents include, but are not limited to, corticosteroids such as synthetic glucocorticoids. Specific examples are fluticasone, COX-2 inhibitors, ibuprofen, hydroxychloroquine, heparin, LMW heparin, hirudine, or immunosuppressants, such as azathioprine, cyclosporin A, or cyclophosphamide.

Specifically, the calixarene compound is combined with an antibiotic such as a beta lactam antibiotic, an aminoglycoside antibiotic, an ansamycin, a carbacephem, a carbapenem, a cephalosporin, a glycopeptide, a lincosamide, a lipopeptide, a macrolide, a monobactam, a nitrofuran, an oxazolidinone, a polypeptide, a sulfonamide, Clofazimine, Dapsone, Capreomycin, Cycloserine, Ethambutol, Ethionamide, Isoniazid, Pyrazinamide, Rifampicin, Rifabutin, Rifapentine, Streptomycin, Arsphenamine, Chloramphenicol, Fosfomycin, Mupirocin, Platensimycin, Quinupristin/Dalfopristin, Thiamphenicol, Tigecycline, Tinidazole, Trimethoprim, Teixobactin, Malacidins, Halicin, clindamycin, vancomycin, metronidazole, fusidic acid, thiopeptides, fidaxomicin, quinolons, tetracyclins, omadacycline, rifamycin, kibdelomycin, oxazolidinone, ketolides, thiazolides, amixicile, teicoplanin, ramoplanin, oritavancin, lantibiotics, capuramycin, surotomycin, thuricin, endolysin, avidocin CD, cadazolid, ramizol, defensins, ridinilazole, medium-chain fatty acids, phages, berberine, lactoferrin.

Specifically, treatment with the calixarene compound described herein can be combined with a treatment administering at least one other therapeutic agent selected from the group consisting of a corticosteroid, an anti-inflammatory signal transduction modulator, a 2-adrenoreceptor agonist bronchodilator, an anticholinergic, a mucolytic agent, hypertonic saline and other drugs for treating a Coronaviridae virus infections; or mixtures thereof. Specific pharmaceutical compositions may particularly include one or more anti-inflammatory agents, and/or analgesics, PPAR-γ agonists and immune response modulators.

The length of the treatment period depends on a variety of factors, such as the severity of the disease, either acute or chronic disease, the age of the patient, and the concentration of the calixarene compound. It will also be appreciated that the effective dosage used for the treatment or prophylaxis may increase or decrease over the course of a particular treatment or prophylaxis regime. Changes in dosage may result and become apparent by standard diagnostic assays known in the art.

According to a specific aspect, a medicinal product or pharmaceutical composition described herein contains an effective amount of the calixarene compound as defined herein. The preparation described herein may be provided for single or multiple dosage use.

Unit-dose or multi-dose containers may be used, for example, sealed ampoules and vials, or multi-use sprays, and may be stored comprising a liquid or dry phase, e.g., in a freeze-dried (lyophilized) condition requiring only the addition of the sterile liquid carrier, for example water for injection, immediately prior to use. Preferred unit dosage formulations are those containing a daily dose or unit daily sub-dose, or multiple doses, of the calixarene compound.

The term “single-dose” as used herein is understood in the following way. A single-dose or amount for single-use is the amount intended for administration that is meant for use in a single subject, such as a patient, either human or animal for a single case/procedure/administration. Packages comprising the single-dose are typically labelled as such by the manufacturer. The single-dose amount is specifically understood as a daily dose for an individual, like a child or adult, to provide an effective amount.

The medicinal product or pharmaceutical composition described herein is specifically provided as human or veterinary medicinal product or pharmaceutical composition. Medicinal products are understood as substances that are used to treat diseases, to relieve complaints, or to prevent such diseases or complaints in the first place. This definition applies regardless of whether the medicinal product is administered to humans or to animals. The substances can act both within or on the body.

The medicinal product or pharmaceutical composition described herein preferably contains one or more pharmaceutically acceptable auxiliaries and is in a pharmaceutical form which allows the active pharmaceutical compound to be administered with high bioavailability. Suitable auxiliaries may be, for example, based on cyclodextrins. Suitable formulations might for example incorporate synthetic polymeric nanoparticles formed of a polymer selected from the group consisting of acrylates, methacrylates, cyanoacrylates, acrylamides, polylactates, polyglycolates, polyanhydrates, polyorthoesters, gelatin, albumin, polystyrenes, polyvinyls, polyacrolein, polyglutaraldehyde and derivatives, copolymers and mixtures thereof.

Specific medicinal products or pharmaceutical compositions described herein comprise the calixarene compound and a pharmaceutically acceptable carrier or excipient. A “pharmaceutically acceptable carrier” refers to an ingredient in a formulation for medicinal or medical use, other than an active ingredient, which is nontoxic to a subject. A pharmaceutically acceptable carrier includes, but is not limited to, a buffer, excipient, stabilizer, or preservative, and in particular saline, phosphate buffered saline, dextrose, glycerol, ethanol, and the like.

The calixarene compound as used herein can be formulated with conventional carriers and excipients, which will be selected in accord with ordinary practice.

Pharmaceutically acceptable carriers generally include any and all suitable solvents, dispersion media, coatings, antiviral, antibacterial and antifungal agents, isotonic and absorption delaying agents, and the like that are physiologically compatible with an antiviral small molecule compound or related composition or combination preparation described herein.

According to a specific aspect, the calixarene compound can be combined with one or more carriers appropriate a desired route of administration. The calixarene compound may be e.g., admixed with any of lactose, sucrose, starch, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, polyvinyl alcohol, and optionally further tableted or encapsulated for conventional administration. Alternatively, the calixarene compound may be dispersed or dissolved in saline, water, polyethylene glycol, propylene glycol, carboxymethyl cellulose colloidal solutions, ethanol, corn oil, peanut oil, cotton seed oil, sesame oil, tragacanth gum, and/or various buffers. Other carriers, adjuvants, and modes of administration are well known in the pharmaceutical arts. A carrier may include a controlled release material or time delay material, such as glyceryl monostearate or glyceryl distearate alone or with a wax, or other materials well-known in the art.

Compounds as described herein may be provided in controlled release pharmaceutical (“controlled release formulations”) in which the release of the calixarene compound is controlled and regulated to allow less frequency dosing or to improve the pharmacokinetic or toxicity profile of a given active ingredient.

Pharmaceutical compositions may also be coated by conventional methods, typically with pH or time-dependent coatings, such that the subject agent is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac.

Additional pharmaceutically acceptable carriers are known in the art and described in, e.g., Remington: The Science and Practice of Pharmacy, 22^{nd} revised edition (Allen Jr, L V, ed., Pharmaceutical Press, 2012). Liquid formulations can be solutions, emulsions or suspensions and can include excipients such as suspending agents, solubilizers, surfactants, preservatives, and chelating agents.

The preferred preparation is in a ready-to-use, storage stable form, with a shelf-life of at least one or two years.

The term “formulation” as used herein refers to a preparation ready-to-use in a specific way. Specifically, compositions described herein comprises the calixarene compound, and a pharmaceutically acceptable diluent, carrier or excipient.

According to a specific aspect, formulations are provided comprising pharmaceutically acceptable vehicles for nasal, intrapulmonary, oral, topical, mucosal or parenteral administration. Administration may also be intradermal or transdermal. Also, the present disclosure includes such compounds, which have been lyophilized and which may be reconstituted to form pharmaceutically acceptable formulations for administration.

Specific medicinal products or pharmaceutical compositions described herein are formulated for intranasal administration or by another topical route e.g., onto biological surfaces, including e.g., mucosa or skin. Pharmaceutical carriers suitable for facilitating such means of administration are well known in the art.

Specifically, a nasal spray may be used containing 0.1% or 0.15% (w/w) calixarene compound in an aqueous solution at pH 6.8±0.3, optionally further containing any one or more of citric acid monohydrate, disodium hydrogen phosphate dodecahydrate, edetate disodium, hypromellose, purified water, sodium chloride, and a preservative such as benzalkonium chloride.

To administer the calixarene compound by any route other than parenteral administration, it may be necessary to coat the active agent with, or co-administer the active agent with, a material to prevent its inactivation. For example, an appropriate carrier may be used, for example, liposomes, or a diluent. Pharmaceutically acceptable diluents include saline and aqueous buffer solutions.

The calixarene compound can be orally administered, for example, with an inert diluent or an assimilable or edible carrier. For example, a preparation may be enclosed in a hard- or soft-shell gelatin capsule, or compressed into tablets. For oral therapeutic administration, the calixarene compound may be incorporated with excipients and used in the form of ingestible tablets, buccal tablets, troches, capsules, elixirs, suspensions, syrups, wafers, and the like. The percentage of the compound in the compositions and preparations may, of course, be varied. The amount of the calixarene compound in such therapeutically useful compositions is such that a suitable dosage will be obtained.

Tablets will contain excipients, glidants, fillers, binders, disintegrants, lubricants, flavors and the like. Granules may be produced using isomaltose. It is furthermore preferred to provide for a preparation formulated to act at the site of the mucosa, e.g. at mucosal sites (such as nose, mouth, eyes, esophagus, throat, lung), e.g. locally without systemic action. Aqueous formulations are prepared in sterile form, and when intended for delivery by other than oral administration generally will be isotonic.

The term “mucosal” with respect to administration or application or else mucosal use of a preparation for treating a subject or a respective formulation, refers to administration via the mucosal route, including systemic or local administration, wherein an active ingredient is taken up by contact with mucosal surfaces. This includes nasal, pulmonary, oral, or peroral administration and formulations, e.g. liquid, syrup, lozenge, tablet, spray, powder, instant powder, granules, capsules, cream, gel, drops, suspension, or emulsion.

Peroral formulations may include liquid solutions, emulsions, suspensions, and the like. The pharmaceutically acceptable vehicles suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, tragacanth, and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Other compositions useful for attaining systemic delivery of the calixarene compound or respective preparations include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose, or glidants, lubricants, sweeteners, colorants, antioxidants and flavoring agents.

The calixarene compound or respective preparations can also be administered topically to a subject, e.g., by the direct laying on or spreading of a composition containing same on the epidermal or epithelial tissue of the subject, or transdermally via a “patch”. Such compositions include, for example, lotions, creams, solutions, gels and solids. These topical compositions may comprise an effective amount, usually at least about 0.1 wt %, or even from about 1 wt % to about 5 wt %, of the calixarene compound. Suitable carriers for topical administration typically remain in place on the skin as a continuous film, and resist being removed by perspiration or immersion in water. Generally, the carrier is organic in nature and capable of having dispersed or dissolved therein the therapeutic agent. The carrier may include pharmaceutically acceptable emollients, emulsifiers, thickening agents, solvents and the like.

Pharmaceutical compositions suitable for injectable use include sterile aqueous solutions (in particular where the compounds or pharmaceutically acceptable salts are water soluble) or dispersions and sterile powders for the extemporaneous preparation of sterile injectable solutions or dispersion. In particular, the composition is specifically sterile and fluid to the extent that easy syringability exists; it is stable under the conditions of manufacture and storage and preserved against the contaminating action of microorganisms such as bacteria and fungi.

Suitable pharmaceutically acceptable vehicles include, without limitation, any non-immunogenic pharmaceutical adjuvants suitable for oral, parenteral, nasal, mucosal, transdermal, intravascular (IV), intraarterial (IA), intramuscular (IM), and subcutaneous (SC) administration routes, such as phosphate buffer saline (PBS).

The term “subject” as used herein shall refer to a warm-blooded mammalian, particularly a human being or a non-human animal, including e.g., dogs, cats, rabbits, horses, cattle, and pigs. In particular the treatment and medical use described herein applies to a subject in need of prophylaxis or therapy of a disease condition associated with a coronavirus infection. Specifically, the treatment may be by interfering with the pathogenesis of a disease condition where a coronavirus is a causal agent of the condition. The subject may be a patient at risk of such disease condition or suffering from disease.

The term “at risk of” a certain disease conditions, refers to a subject that potentially develops such a disease condition, e.g. by a certain predisposition, exposure to virus or virus-infected subjects, by exposure to a certain disease causing agent such as a virus, or that already suffers from such a disease condition at various stages, particularly associated with other causative disease conditions or else conditions or complications following as a consequence of viral infection. The risk determination is particularly important in a subject, where a disease has not yet been diagnosed. This risk determination therefore includes early diagnosis to enable prophylactic therapy. Specifically, the calixarene compound is used in subjects with a high risk, e.g. a high probability of developing disease.

The term “patient” includes human and other mammalian subjects that receive either prophylactic or therapeutic treatment. The term “patient” as used herein always includes healthy subjects. The term “treatment” is thus meant to include both prophylactic and therapeutic treatment.

Specifically, the term “prophylaxis” refers to preventive measures which is intended to encompass prevention of the onset of pathogenesis or prophylactic measures to reduce the risk of pathogenesis.

The term “therapy” as used herein with respect to treating subjects refers to medical management of a subject with the intent to cure, ameliorate, stabilize, reduce the incidence or prevent a disease, pathological condition, or disorder, which individually or together are understood as “disease condition”. The term includes active treatment, directed specifically toward the improvement of a disease condition, prophylaxis directed specifically toward the prevention of a disease condition, and also includes causal treatment directed toward removal of the cause of the associated disease condition. In addition, this term includes palliative treatment designed for the relief of symptoms rather than the curing of the disease condition, and further curing a disease condition directed to minimizing or partially or completely inhibiting the development of the associated disease condition, and supportive treatment employed to supplement another specific therapy directed toward the improvement of the associated disease condition.

The foregoing description will be more fully understood with reference to the following examples. Such examples are, however, merely representative of methods of practicing one or more embodiments of the present invention and should not be read as limiting the scope of invention.

### EXAMPLES

### Example 1: Preventing SARS-CoV-2 Infection of Cells by CX-1

To detect the effect of CX-1 (4-Sulfocalix[4]arene, Sigma Aldrich Cat #55523, Merck KGaA, Darmstadt, Germany) on SARS-CoV-2 infection, Vero E6 (monkey kidney) cells were infected with SARS-CoV-2 (Source: isolated by the Virology Laboratory, University of Pecs, Hungary The test was performed in two different settings. 1., virus infection was done in the presence of CX-1 or 2., cells were first infected with the virus, then treated with CX-1. The effectiveness of CX-1 to prevent infection was evaluated by microscopic examination of the cells and virus quantification was done by RT-PCR analysis or droplet PCR analysis of culture supernatants.

### Experimental Procedure

Vero E6 cells (ATCC CRL-1586) were seeded on 96-well plates. After 2 days the cell cultures reached confluency and formed a homogenous monolayer. The cells were fed with fresh cell culture medium and infected with SARS-CoV-2 at MOI 0.01 (multiplicity of infection: 1 viral particle to 100 cells). In the “prophylactic setting” virus infection was done in the presence of CX-1 and after 30 min incubation, the culture medium was removed and replaced with fresh culture medium containing CX-1. In the “therapeutic setting” cells were first infected for 30 min, then culture medium was removed and replaced with culture medium containing CX-1. CX-1 was used at 500, 100 or 20 μM or at 300, 200, 100 and 50 μM concentrations in the prophylactic and therapeutic settings, respectively. The virus stock was prepared by propagation in Vero E6 cells and the infectious titre determined by using the plaque assay. 48 hours post infection the cells were evaluated by microscopic observation. Samples from the cell culture supernatant were taken at 24 and 48 hours for quantitative RT-PCR analysis. RNA was extracted with standard methods, and for viral copy number quantification droplet digital PCR technology was applied (Bio-Rad Laboratories Inc. QX200 Droplet Digital PCR System). The primers and probes used were specific for the SARS-CoV-2 RdRp gene (Reverse primer: CARATGTTAAASACACTATTAGCATA (SEQ ID NO:2), Forward primer: GTGARATGGTCATGTGTGGCGG (SEQ ID NO:3), Probe: FAM-CAGGTGGAACCTCATCAGGAGATGC-BBQ (SEQ ID NO:4)). The droplet PCR kit was used (BioRad ddPCR™, Bio-Rad Laboratories GmbH, Germany). The results of the RT-PCR reaction were quantified and calculated as viral particle/μl.

Results:

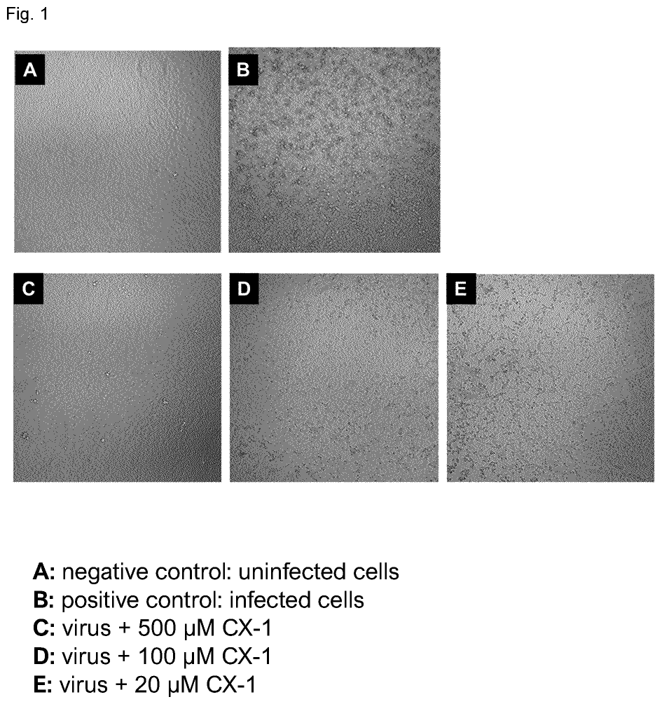

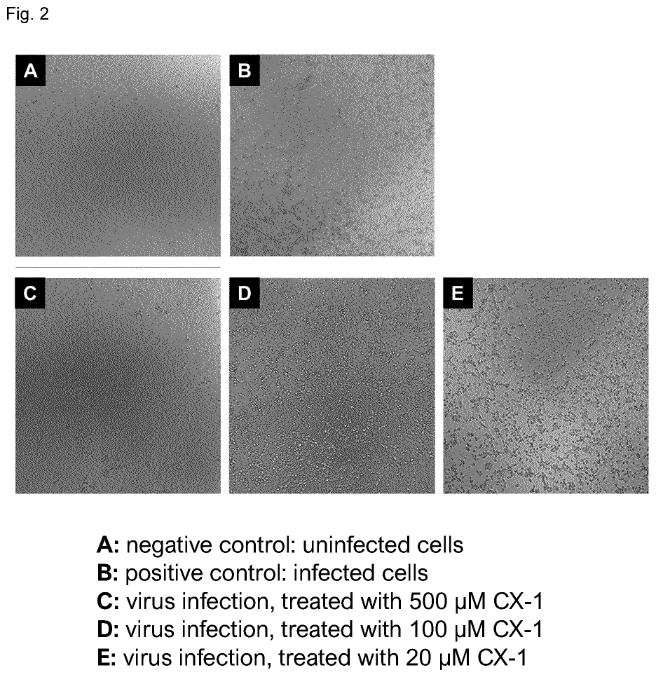

The infection and the cytopathogenic effect of the SARS-CoV-2 virus is detected by microscopic examination of the cells. The confluent, homogenous layer of cells is disrupted and “holes” appear indicating cell death due to the virus. In the presence of CX-1, the SARS-CoV-2 infected cells were significantly protected from dying providing evidence of direct anti-viral effect in both the prophylactic (FIG. 1) and therapeutic settings (FIG. 2). The protective effect was more prominent in the prophylactic setting and dependent on the concentration of CX-1 and was the most prominent at the highest concentration (500 μM) (FIG. 1). In the therapeutic setting, it was mainly the highest concentration of CX-1 that was effective (FIG. 2). These data suggest that CX-1 was efficient to prevent the infection of cell by SARS-CoV-2 both at the first encounter and after the virus already have propagated inside cells and started infecting other cells.

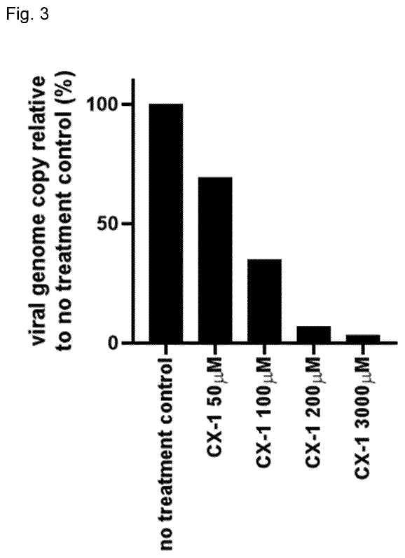

The protection from viral infection was further proven by quantitative PCR analysis of the reverse transcribed viral RNA. At 24 and 48 hours, post-infection in the prophylactic setting, an approximately 82% and 95% reduction in viral particles was observed in the presence of 500 μM CX-1 (Table 1). Upon testing lower dilutions of CX-1 in the preventive setting, we observed over 90% of reduction in viral genome copy number compared to infected, but non-treated control and even 50 μM of CX-1 was effective in reducing viral genome copy number by 30% (FIG. 3).

TABLE 1
Results of PCR quantification of viral particle numbers in
cell culture supernatants of FIG. 1; B: virus infected and
C: virus infected in the presence of CX-1 500 μM at 24
and 48 hours post-infection in triplicate measurements.

| | | Sample | Sample | Sample | | |
|---|---|---|---|---|---|---|
| | | #1 | #2 | #3 | average | potency |
| 24 h | virus | 2.34 | 3.73 | 1.94 | 2.67 | |
| | virus + CX-1 | 0.00 | 0.00 | 1.48 | 0.49 | 82% |
| 48 h | virus | 229.00 | 156.00 | 255.00 | 213.33 | |
| | virus + CX-1 | 11.10 | 15.00 | 8.63 | 11.57 | 95% |

values represent viral particle number/μl cell culture supernatant

### Example 2. Preventing Infection of MucilAir™ by Influenza a Virus H1N1

The effect of repeated dose CX-1 upon influenza H1N1 infection was tested on fully differentiated human nasal epithelial cells cultured at the air-liquid interface (MucilAir™ Pool, Epithelix Sarl-primary cells from a pool of 14 different normal nasal donors).

### Experimental Procedure

Antiviral effect of CX-1 against influenza H1N1 was tested as described by Boda et al (Antiviral Research 156 (2018) 72-79). Briefly, following apical wash of MucilAir™ Pool with MucilAir™ culture media (200 μl for 10 min), 10 μl of CX-1 (4-Sulfocalix[4]arene, Sigma Aldrich Cat #55523, Merck KGaA, Darmstadt, Germany) at 500 μM concentration was applied to the apical side of MucilAir™ for 10 minutes. Immediately afterwards, 100 μl of influenza H1N1 (ATCC VR-95), from a stock of 10⁶ genome copy number per ml) was applied on the apical side and incubated for 3 h. The inoculum was removed by washing the apical side of the cells with 200 μl of MucilAir culture media (20 min incubation) thrice. After the 3^{rd} wash, the same concentration of CX-1 was added to the apical side of the cells at 10 μl volume and incubated for 21 h. At 24 h, 48 and 72 h timepoints MucilAir™ cell washes were repeated after which CX-1 was added on the apical side as before. The basal culture medium was also removed and replaced with 500 μl of fresh culture medium, daily. From the basal wash LDH release was measured (to assess cell death at 96 h). All incubation steps were carried out at 34° C. at 5% CO₂ and at 100% humidity. Viral copy number was determined from the apical washes: for this RNA was extracted with the QIAamp® Viral RNA kit (Qiagen) and the viral RNA was quantified by RT-PCR (QuantyTect Probe RT-PCR, Qiagen) with the qTOWER3 detection system. Ct data were reported normalized to the standard curve and expressed as genome copy number/ml.

Cilia beating frequency was measured on day 4 post-infection with a dedicated system. The system consists of three parts: a camera connected to a microscope, a PCI card and a specific package of software based on Fourier spectral analysis. The Cilia beating frequency is expressed as Hz. Two hundred and fifty-six images of a movie recorded from each well containing tissues were captured at high frequency rate (125 frames per second) and cilia beating frequency was calculated using a Cilia-X software (Epithelix San, France).

As negative control, uninfected cells were included. As positive control for infection, cells infected with influenza H1N1 but treated with buffer only were used). Antiviral effect of CX-1 was compared to the effect of oseltamivir carboxylate (oseltamivir from Carbosynth (Compton, UK) at 10 μM) added to infected cells in the basolateral compartment. Compounds were tested in triplicates.

Results:

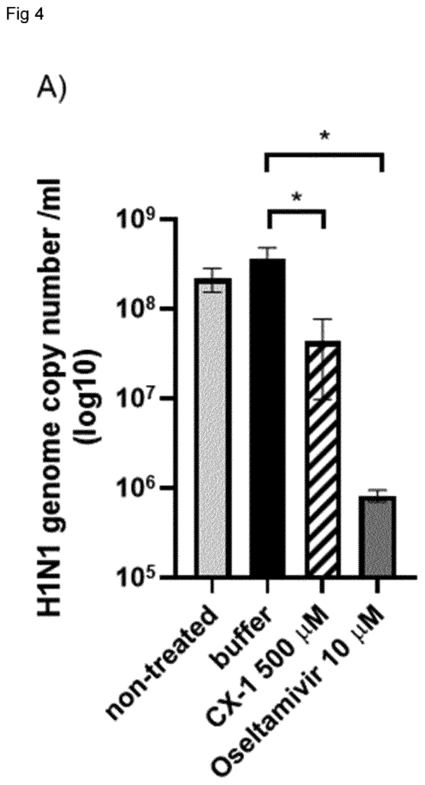

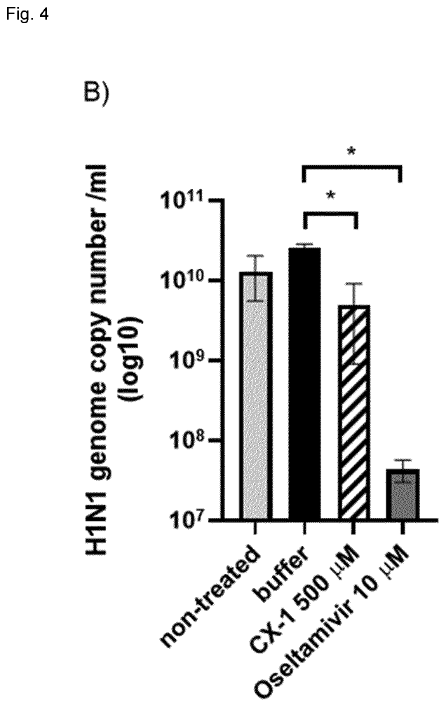

Treatment of MucilAir™ cells with CX-1 resulted in a statistically significantly decrease in viral genome copy at 24 h post-infection, compared to buffer treated cells. The decrease in the viral copy was −0.9 log (88% reduction) at 24 h (FIG. 4 panel A) and ˜0.8 log (80.5% reduction) at 48 h post infection (FIG. 4 panel B).

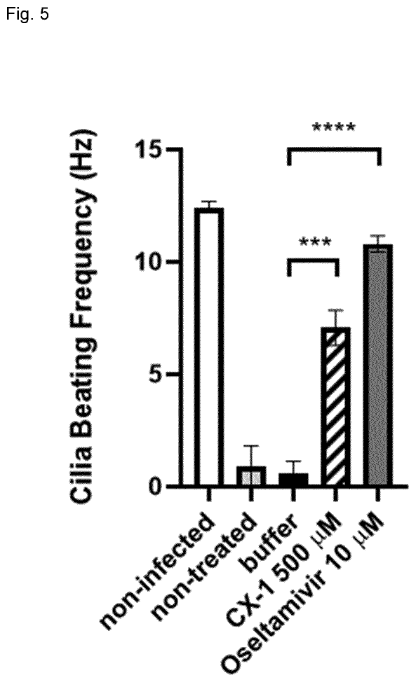

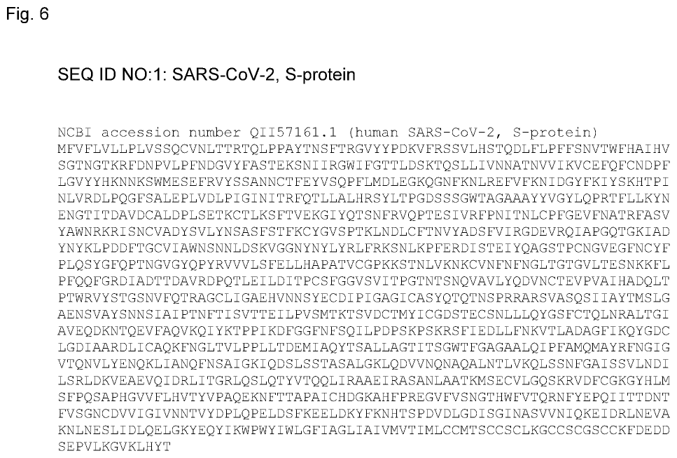

Additionally, CX-1 treatment preserved the active cilia beating on the epithelial cells, the cilia beating frequency (Hz) on day 4 post infection was significantly higher on CX-1 treated cell, as on buffer treated cells (FIG. 5). CX-1 showed no cytotoxicity in non-infected cells.

1. A method for prophylactically or therapeutically treating a disease condition which is caused by or associated with an infection of a subject with one or more viruses comprising at least one respiratory virus, comprising administering an antiviral effective amount of a calixarene compound to the subject.

2. The method of claim 1, wherein said one or more viruses are selected from:
a) a Coronaviridae virus;
b) an Adenoviridae virus;
c) a Paramyxoviridae virus; or
d) an Orthomyxoviridae virus.

3. The method of claim 1, wherein said one or more viruses comprise a coronavirus.

4. The method of claim 1, wherein said one or more viruses comprise any one or both of SARS-CoV-2 and influenza virus.

5. The method of claim 1, wherein the calixarene compound is formulated as a pharmaceutical preparation in admixture with a pharmaceutically acceptable carrier.

6. The method of claim 1, wherein the disease condition is common cold, infection of the nose, sinusitis, throat and larynx, bronchiolitis, diarrhea, rash on skin, or pneumonia, acute respiratory distress syndrome (ARDS).

7. The method of claim 1, wherein the antiviral effective amount is effective in preventing infection of susceptible cells by the virus, thereby treating the disease condition.

8. The method of claim 1, wherein the antiviral effective amount is administered locally or systemically.

9. The method of claim 1, wherein the calixarene compound is a calix[n]arene characterized by the structure (I): 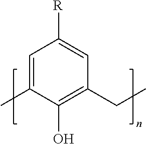
wherein n=denotes 4, 5, 6, 7, or 8 and
R is selected from the group consisting of —SO₃R^{a}, ORE, COOR^{a}, hydrogen, or halogen, and
each R^{a} is hydrogen or methyl;
or a pharmaceutically acceptable salt thereof.

10. The method of claim 9, wherein the calix[n]arene is a calix[4]arene with four intrannular hydroxyl groups on the lower rim and four sulfonate or carboxyilate groups on the upper rim.

11. The method of claim 5, wherein said pharmaceutical preparation is formulated for local administration to the upper and lower respiratory tract or by nasal, pulmonary, intraoral, ocular, or dermal delivery, or is formulated for systemic administration by intravenous, intramuscular, subcutaneous, intradermal, transdermal, or oral administration.

12. The method of claim 5, wherein said pharmaceutical preparation is administered to the subject as a spray, a powder, a gel, an ointment, a cream, a foam, or a liquid solution, a lotion, a gargle solution, an aerosolized powder, an aerosolized liquid formulation, granules, capsules, drops, tablet, syrup, lozenge, or a preparation for infusion or injection.

13. The method of claim 1, wherein the calixarene compound is applied into the subject's nose in an antiviral effective amount of 1-1000 μg per nostril.

14. The method of claim 1, wherein the calixarene compound is administered as the sole antiviral substance, or wherein treatment is combined with a further treatment with one or more active substances.

15. The method of claim 1, wherein the subject has been infected or is at risk of being infected with said virus.

16. A method for treating a biological surface to prevent virus infection and/or virus spread, comprising applying an antiviral effective amount of a calixarene compound to the biological surface preferably wherein said medicinal product is formulated for topical use, such as application to the upper and lower respiratory tract, nasal, pulmonary, intraoral, ocular, or dermal application.

17. The method of claim 2, wherein said one or more viruses are selected from:
a) SARS-CoV-2, MERS-CoV, SARS-CoV-1, HCoV-OC43, HCoV-HKU1, HCoV-NL63, HCoV-229E, PEDV, or naturally-occurring variants or mutants of any of the foregoing;
b) HAdVB, HAdVC, or HAdVD;
c) RSV; or
d) an influenza virus.

18. The method of claim 10, wherein the calix[n]arene is Sulfonatocalixarene or Sulfonatocalixarene sodium salt.

19. The method of claim 14, wherein the one or more active substances are selected from the group consisting of an antiviral substance, an anti-inflammatory substance, and an antibiotic substance.

20. The method of claim 16, wherein said calixarene compound is formulated for topical use for application to the upper and lower respiratory tract, or for nasal, pulmonary, intraoral, ocular, or dermal application.


SEQUENCE LISTING (4 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 26 bp, Artificial Sequence
  Primer
caratgttaaasacactattagcata

SEQ ID NO: 3 - DNA, 22 bp, Artificial Sequence
  Primer
gtgaratggtcatgtgtggcgg

SEQ ID NO: 4 - DNA, 25 bp, Artificial Sequence
  Probe
caggtggaacctcatcaggagatgc